(19) United States
(12) Patent Application Publication
(10) Pub. No.: US 2008/0214938 A1
(43) Pub. Date: Sep. 4, 2008

(54) Optimized Temperature Measurement in an Ultrasound Transducer

(75) Inventors: Rodney J. Solomon, Lowell, MA (US); Jacquelyn Byron, Arlington, MA (US); Heather Knowles, Devens, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/993,893

(22) PCT Filed: Jun. 15, 2006
(86) PCT No.: PCT/IB2006/051937

§ 371 Date: Dec. 26, 2007

Related U.S. Application Data
(60) Provisional application No. 60/695,044, filed on Jun. 29, 2005.

(51) Int. Cl. A61B 8/00

(52) U.S. Cl. 600/459

(57) ABSTRACT

There is provided a medical ultrasound transducer (130) and a medical ultrasound imaging system including the transducer (110, 130, 310, 320, 330, 340, 350), comprising an acoustic window (120) for contacting a patient at a patient contact surface for imaging the patient; and at least one temperature sensor (110, 110A, 110B) located in the acoustic window to determine patient contact temperature at the patient contact surface. The medical ultrasound imaging system further comprises a controller (310) for controlling a power imaging mode of the ultrasound transducer in accordance with the determined patient contact temperature. Also provided is a method for imaging a patient using the medical ultrasound imaging system, comprising contacting the patient contact surface of the acoustic window to the patient for imaging the patient; determining patient contact temperature of the ultrasound transducer at the patient contact surface from the at least one temperature sensor; and controlling a power imaging mode of the ultrasound transducer in accordance with the determined patient contact temperature.

The present invention relates generally to ultrasound medical transducers. More particularly, the present invention is directed to an ultrasound medical transducer having optimized temperature measurement, an ultrasound system and ultrasound patient safety feedback methods therefor.

An ultrasound medical transducer (the “transducer”) is used to observe the internal organs of a patient. The ultrasound range of the transducer is described essentially by its lower limit: 20 kHz, roughly the highest frequency a human can hear. The transducer emits ultrasound pulses which echo (i.e., reflect), refract, or are absorbed by structures in the patient's body. The reflected echoes are received by the transducer and the received signals are translated into images displayed on a video device. Such translation is possible because the reflections from the internal organs vary in intensity according to the “acoustic impedance” between adjacent structures. The acoustic impedance of a tissue is related to its density; the greater the difference in acoustic impedance between two adjacent tissues the more reflective will be their boundary.

The frequency of the ultrasound beams has an effect on both the image resolution and the penetration ability of the transducer. Higher frequency ultrasound waves have a longer near field (i.e., the region in the sound beam's path where the beam diameter decreases as the distance from the transducer increases) and less divergence in the far field (i.e., the region in the sound beam's path where the beam diameter increases as the distance from the transducer increases). Higher frequency ultrasound waves thus permit greater resolution of small structures. However, high-frequency ultrasound waves have less penetrating ability because their energy is absorbed and scattered by soft tissues in the patient's body. On the other hand, lower frequency ultrasound waves have a greater depth of penetration, but the received images are much less well defined. The conventional frequency range for imaging human internal organs (using sound waves) is typically from about 3 MHz to about 5 MHz.

Two types of resolution generally apply: lateral resolution and axial resolution. Lateral resolution is the ability to resolve objects side by side and, as discussed above, is proportionally affected by the frequency (the higher the frequency, the higher the lateral resolution). Higher frequency transducers are used for infants and children because there is less need for deep penetration and the smaller structures can be viewed with greater lateral resolution. Lower frequencies are used for adults where the internal structures are larger and there is a greater need for depth penetration. Of course, when determining the appropriate frequency to be used, the structure, tissue, or organ to be viewed (and the exact purpose of the imaging) can matter more than the age of the subject. For example, diagnostic breast imaging on an adult may require a frequency of about 7 MHz or higher. Axial resolution is the ability to resolve objects that lie one above the other. Because this is related to depth penetration, axial resolution is inversely proportional to the frequency of the transducer (depending on the size of the patient). In large patients, higher frequency beams are rapidly absorbed by the objects closest to the transducer, thus reducing depth penetration and axial resolution.

It is desirable for the ultrasound system to operate at the highest frequency (for the reasons discussed above) and at the maximum acoustic intensity. Maximizing the acoustic intensity increases imaging performance by increasing the depth penetration and maximizing the signal to noise ratio (SNR). However, higher frequencies and greater acoustic intensities cause the transducer to heat up, and there are regulatory limits (and practical limits) on the surface temperature of the transducer interacting with a patient. Specifically, the upper temperature limit on the patient contact surface of the transducer is generally considered to be about 41° C. or about 16° C. above the ambient temperature.

The heat of the transducer surface is generated both by the electroacoustic energy conversion process that takes place in the transducer's piezoelements and by the acoustic energy that passes through and/or into adjacent transducer materials (and the patient). Different methods and systems have been developed to deal with the heating problem and they can be broken into two types: active and passive. Passive solutions use cooling mechanisms, i.e., spreading out the dissipated heat to as large an external transducer surface area as possible. Typically, the heat generated by the transducer array is absorbed by solid thermal conductors, and then this captured heat is moved by thermal convection into the transducer's external case, where it can dissipate in the ambient atmosphere. Ideally, the external heat-convecting surface area would consist of the transducer's entire external surface area.

One example of a passive heat dissipation system is U.S. Pat. No. 5,213,103 (the '103 patent), which is hereby incorporated by reference in its entirety. A heat sink device which is internal to the transducer is placed inside the transducer 10 behind the piezoelectric elements in the face 14 (i.e., the patient contact surface) on the head 12 of transducer 10. The heat sink extends the entire length of the transducer and conducts heat away from face 14, through head 12, to the sides of handle 16 and power cable 18. Heat conductive epoxy is used both to attach the heat sink to the transducer housing and to conduct the heat from the heat sink to the transducer housing.

Another example of a passive heat dissipation system is U.S. Pat. No. 5,555,887 (the '887 patent), which is hereby incorporated by reference in its entirety. The '887 patent applies heat dissipation to an endoscopic ultrasound transducer by embedding aluminum foil in acoustic lens material in front of the transducer array. Heat is conducted by the aluminum foil to a heat sink positioned at a distance from the patient contact surface of the probe. U.S. Pat. No. 5,721,463 (the '463 patent), which is hereby incorporated by reference in its entirety, describes a passive heat dissipation system which uses a bundle of coaxial cables to vent heat away from the face of the probe.

These passive heat sinks are effective, but they also add to the transducer's overall thermal dissipation resistance. The fundamental limitation is that, for most transducers, even if heat is spread uniformly on the external case surfaces, it only takes a few watts of transducer driving power to cause the average transducer surface temperature to become unacceptably high either with respect to the patient or the sonographer. In these cases, and particularly for small transducers having small surface areas, one may find that one is unable to operate at the allowable acoustic intensity limit because of excessive temperatures.

Active solutions, on the other hand, use active cooling means, such as circulating coolant systems. One example, U.S. Pat. No. 5,560,362 (the '362 patent), which is hereby incorporated by reference in its entirety, describes a heat dissipation system in which a pumping or pressurization means actively circulates a gaseous or liquid coolant in a cable, part of which is nearby the transducer array. The system can be a single pass, multipass, or closed loop circulating system, and the coolant may pass through a heat exchanger, a heat pipe, a thermoelectric cooler, an evaporator/condenser system, and/or a phase change material.

Several different ultrasound transducer cooling systems exist, which monitor or control the temperature of the transducer using feedback from a temperature sensor placed within or next to the transducer. If the patient contact surface temperature is determined to be at or near a safety threshold temperature, the power into the transducer will be limited in order to decrease the amount of heat, which in turn negatively impacts the ultrasound image quality. It is desirable to measure the patient contact surface temperature as precisely as possible so that the transducer can be operated at the highest power available to produce best possible image quality.

An ultrasound transducer cooling system which uses feedback control is shown in U.S. Pat. No. 6,210,356 (the '356 patent), which is hereby incorporated by reference in its entirety. The '356 patent is directed to a catheter which provides ultrasound energy (and perhaps medicine) as a therapeutic treatment to a site inside a patient's body. Thus, no imaging or sensing is being performed by the ultrasound transducer in the '356 patent. Temperature sensors are positioned in the surface coating of the catheter next to the ultrasound transducer in order to provide a measure of the temperature on the exterior surface of the catheter. This measure is used as a feedback control signal for the power circuits of the ultrasound transducer. After the user sets a predetermined temperature, the power circuits decrease or increase power in the same proportion as the measured temperature is above or below the predetermined temperature. The system described in the '356 patent also includes safety control logic which detects when the temperature at a temperature sensor has exceeded a safety threshold. When this occurs, the power circuits stop providing power to the ultrasound transducer. However, such a feedback control system can be inappropriate for ultrasound imaging/measuring applications.

Although turning off the power abruptly during a therapeutic ultrasound session may not be damaging, turning off the power abruptly during an imaging/measuring session can be potentially dangerous (e.g., a sudden blackout during a surgical procedure is dangerous). Even when not dangerous, turning off the imaging makes the diagnosis and analysis of image data more difficult.

The above-described prior art systems have located the temperature sensor within or next to the transducer some distance from the patient contact surface. As such, the temperature at the temperature sensor likely will not be the same as the temperature at the patient contact surface. Algorithms have been developed to predict the patient contact surface temperature. The algorithms are correct for one set of environmental conditions. If the conditions are different from those used to develop the algorithm, the system will not correctly predict the temperature of the patient contact surface temperature.

Notwithstanding the various improvements in ultrasound medical transducers, there is a need in the art to provide an ultrasound medical transducer, system and method with optimized temperature measurement for detecting patient contact surface temperature more precisely in order to provide the best possible image quality while mitigating hazards to patient safety.

The present invention is directed to an ultrasound medical transducer having optimized temperature measurement, an ultrasound imaging system and ultrasound patient safety feedback methods therefor.

According to an embodiment of the present invention, there is provided a medical ultrasound transducer comprising: an acoustic window for contacting a patient at a patient contact surface for imaging the patient; and at least one temperature sensor located in the acoustic window for determining patient contact temperature at the patient contact surface.

According to another embodiment of the present invention, there is provided a medical ultrasound imaging system comprising: an ultrasound transducer including (i) an acoustic window for contacting a patient at a patient contact surface for imaging the patient; and (ii) at least one temperature sensor located in the acoustic window for determining patient contact temperature at the patient contact surface; and a controller for controlling a power imaging mode of the ultrasound transducer in accordance with the determined patient contact temperature.

According to yet another embodiment of the present invention, there is provided a method for imaging a patient using a medical ultrasound imaging system, the imaging system comprising an ultrasound transducer including an acoustic window having a patient contact surface and at least one temperature sensor located in the acoustic window, the method comprising: contacting the patient contact surface of the acoustic window to the patient for imaging the patient; determining patient contact temperature of the ultrasound transducer at the patient contact surface from the at least one temperature sensor; and controlling a power imaging mode of the ultrasound transducer in accordance with the determined patient contact temperature.

The features and advantages of the present invention will become apparent to one skilled in the art, in view of the following detailed description in combination with the attached drawings, in which:

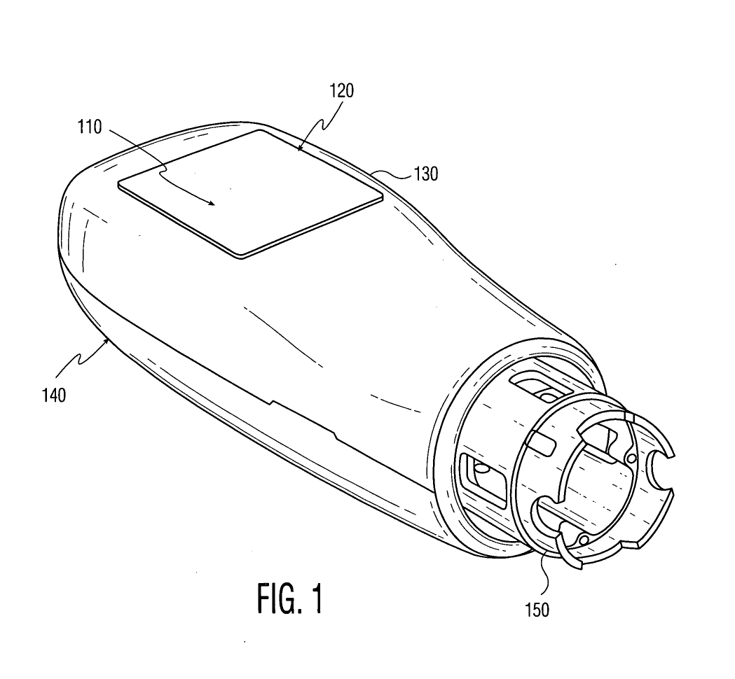

FIG. 1 is an exemplary ultrasound medical transducer of an ultrasound imaging system;

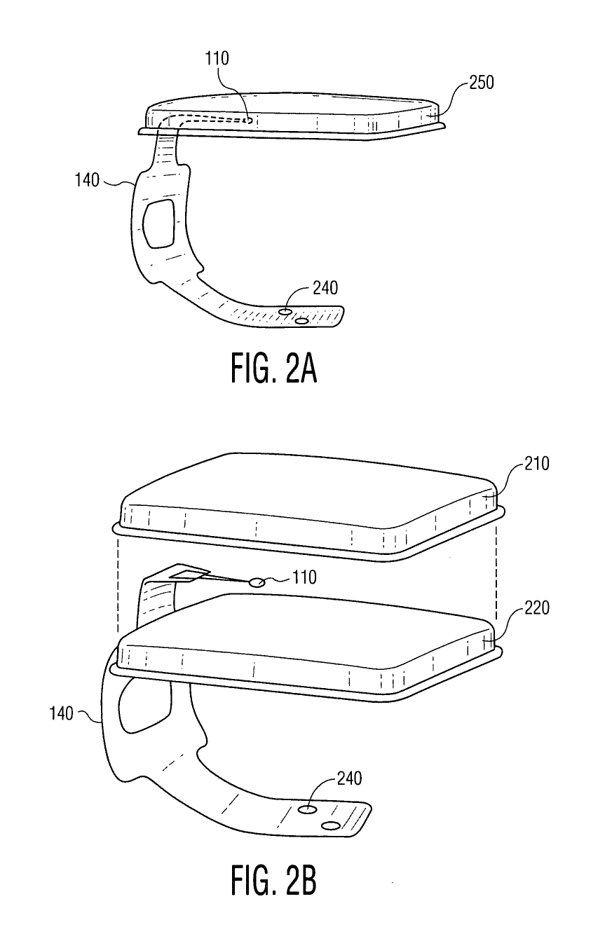

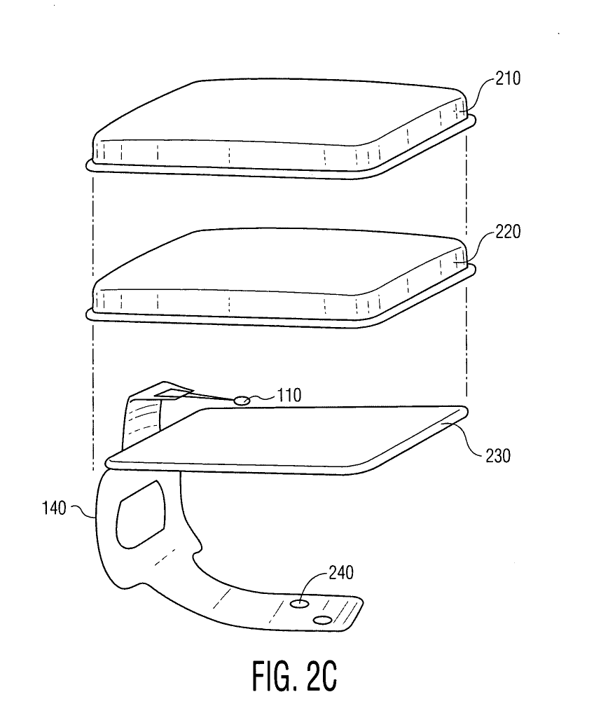

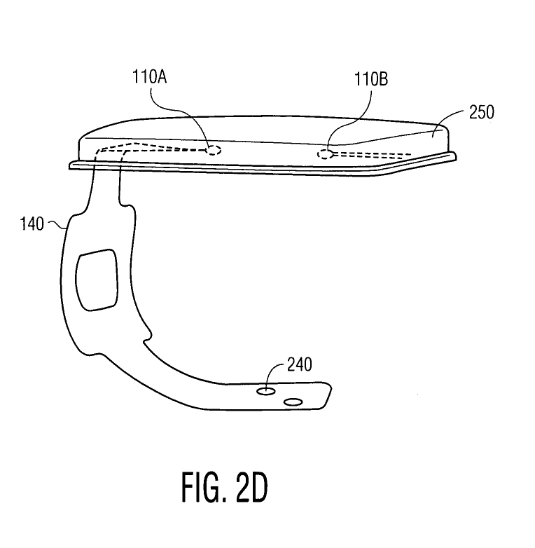

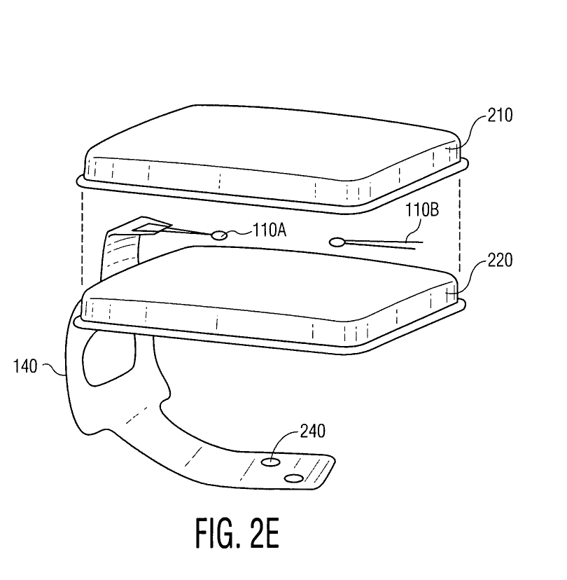

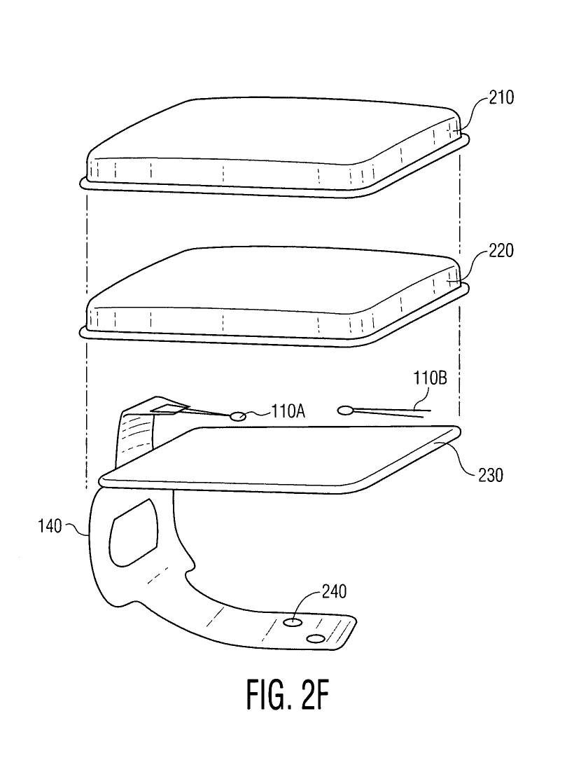

FIGS. 2A-2F are exemplary exploded configurations of the acoustic window depicted in FIG. 1, comprising at least one layer and at least one temperature sensor;

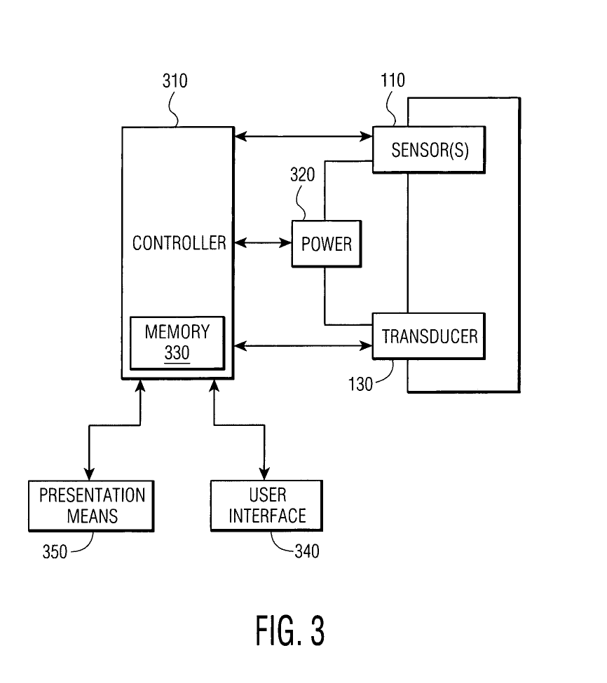

FIG. 3 is an exemplary block diagram of an ultrasound medical imaging system comprising the ultrasound medical transducer depicted in FIG. 1;

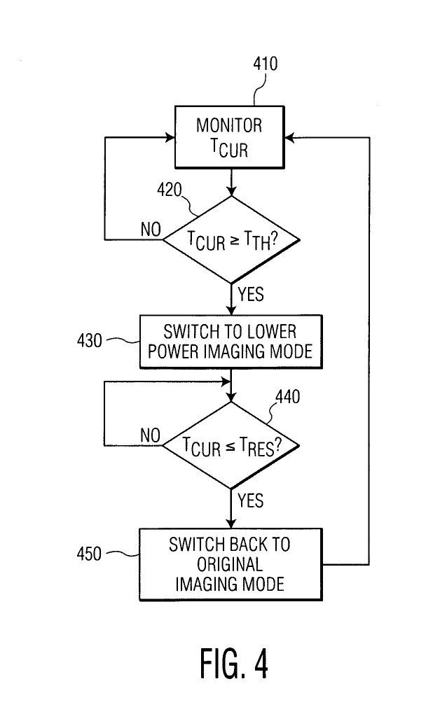

FIG. 4 is an exemplary flowchart of a patient safety feedback method that resets an imaging mode at a predetermined temperature within the ultrasound medical imaging system depicted in FIG. 3;

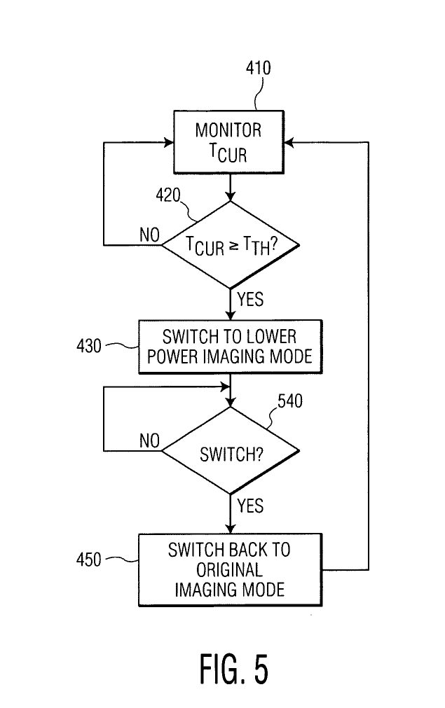

FIG. 5 is an exemplary flowchart of a patient safety feedback method that resets the imaging mode via user input within the ultrasound medical imaging system depicted in FIG. 3;

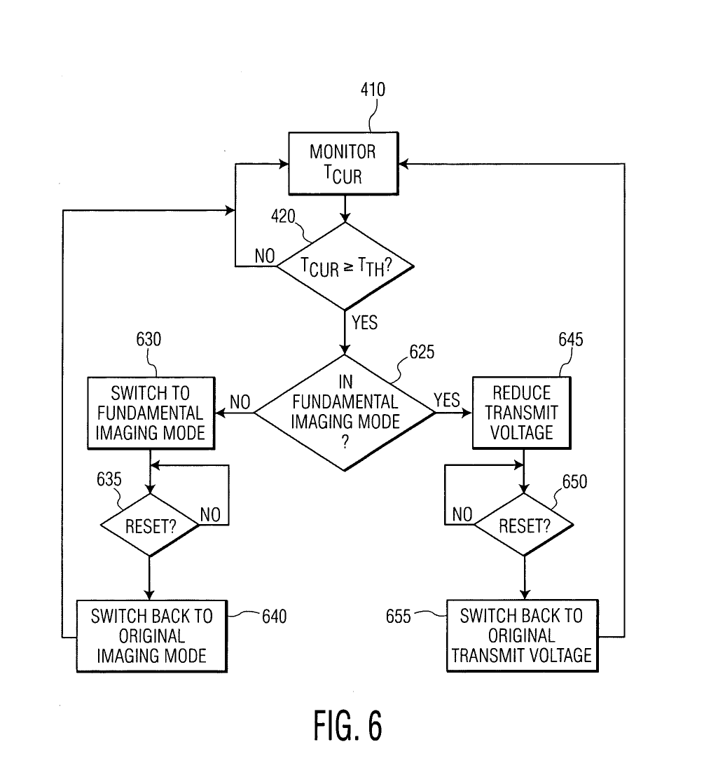

FIG. 6 is an exemplary flowchart of a patient safety feedback method that resets the imaging mode to a fundamental imaging mode or reduces a transmit voltage of the ultrasound medical transducer if the imaging mode is already set to the fundamental imaging mode within the ultrasound medical imaging system depicted in FIG. 3; and

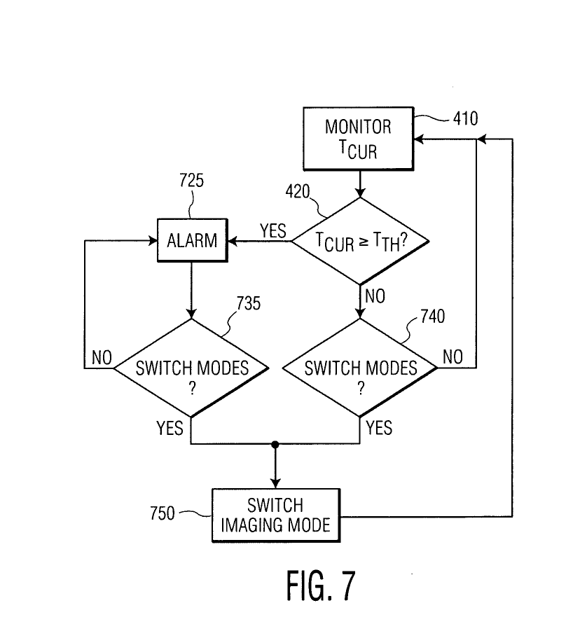

FIG. 7 is an exemplary flowchart of a patient safety feedback method that sets or resets the imaging mode via user input.

The present invention is directed to an ultrasound medical transducer having optimized temperature measurement, an ultrasound imaging system and ultrasound patient safety feedback methods therefor. Although the details of the implementation may be different in different embodiments, the present invention is not limited to any particular type of ultrasound transducer, or any particular mode of imaging. The location of a temperature sensing element (sensor) in the acoustic window of the ultrasound medical transducer in accordance with FIGS. 1-2F, provides for precise measurement of the temperature at the patient contact surface for accurately controlling the temperature of the transducer in accordance with the different patient safety feedback methods of FIGS. 4-7. Each of the functional modules of the ultrasound medical imaging system depicted in FIG. 3 should be understood as an abstraction of the function or combination of functions named, and each can be combined or further divided as necessary for implementing a particular embodiment. These functions may be implemented in software, hardware, or a combination thereof. The ultrasound imaging system may optionally comprise passive or active heat dissipation methods and systems known in the art.

FIG. 1 depicts an exemplary ultrasound medical transducer 130 of an ultrasound imaging system depicted and described in connection with FIG. 3 below. The ultrasound medical transducer 130 (hereinafter “transducer”) comprises at least one temperature sensing element 110 (hereinafter “sensor”) for measuring the temperature and an acoustic window 120 that provides a patient contact surface for imaging the patient. The circuit 140 relays the temperature reading from the sensor 110 to a controller (or processor) that controls the overall operation of the transducer, as will be described in greater detail below with reference to FIGS. 3-7. The interconnect 150 secures the transducer to a cable (not shown) for interconnecting the transducer to the ultrasound imaging system depicted and described in connection with FIG. 3 below. As will be described in greater detail below with reference to FIG. 2A-2F, the sensor 110 (or plurality of sensors 110A, 110B as described with reference to FIGS. 2D-2F) is located in the acoustic window 120 to provide for precise measurement of the temperature at the patient contact surface and to mitigate the hazards to patient safety. The acoustic window 120 may be any of a plurality of configurations and materials that provide proper acoustic properties for transmission of acoustic energy through the acoustic window 120. The configurations of the acoustic window 120 may include one layer, two layer or three-layer configurations as described below with reference to FIGS. 2A-2F. The temperature sensing element may be any sensor for providing a precise temperature reading at the patient contact surface of the acoustic window 120, yet be small enough to mitigate the possible interference with or obstruction of transmission of ultrasound energy through the acoustic window 120, which can negatively or deleteriously affect the acoustic imaging quality. An acceptable size of the sensor 110 or (sensors 110A, 110B of FIGS. 2D-2F) is related to the wavelength of the acoustic energy transmitted through the acoustic window 120. More specifically, it is preferable for the sensor to be a small portion of the wavelength in order to minimize reflection and scattering of energy from the sensor and to mitigate interference with the acoustic image.

Further with reference to FIG. 1, the location of the sensor 110 in the acoustic window 120 is determined based on the requirements for particular ultrasound imaging system. More specifically, there are several locations where the sensor 110 will be most suited for particular applications. The objectives common to all locations are to mitigate interference to the transmission of acoustic energy through the acoustic window 120 and to measure the temperature of the hotspot in the acoustic window 120. Typically the hotspot of the transducer 130 is at the center of the region transmitting the acoustic energy through the acoustic window 120. Locating the sensor 110 at the edge of the acoustic window 120 ensures that the sensor interference with acoustic imaging quality is minimal. Alternatively, locating the sensor 110 in the center of the acoustic window 120 may provide the best overall temperature reading, while posing a minimal risk of interference. The risk of possible interference with acoustic imaging quality is mitigated if the size of the sensor is a small portion of the wavelength of the acoustic energy transmitted through the acoustic window 120. In addition, in a spherical acoustic window configuration, the sensor 110 should be located to avoid the toothbite, interference of acoustic energy from a transducer array (not shown) in the spherical acoustic window configuration. There may be a plurality of off-center locations in the acoustic window 120 that would provide beneficial measurements. For example, there can be an off-center location in the acoustic window 120 in which an ultrasound chip (not shown) produces a hotspot. The measurement of the hotspot ensures that the hottest location of the acoustic window is measured to improve patient safety. In addition, offsetting the sensor 110 from the center offers a benefit when using an ultrasound imaging transducer in a so-called “Steerable CW Mode,” in which one half of the transducer 130 is used to transmit ultrasound energy and the other half is used to receive ultrasound energy. The transmit side would get hotter than the receive side. Therefore, locating the sensor 110 over the transmit side would provide a more precise measurement of the hottest region of the transducer 130.

As aforementioned with reference to FIG. 1, the sensor 110 may be any sensor that mitigates the possible interference with or obstruction of transmission of ultrasound energy through the acoustic window 120. The sensor 110 may be a thermistor, a thermocouple, a resistance temperature detector (RTD), a fiber optic sensor using thermalchromic liquid crystals, or two or more of any of the above sensors (if plurality of sensors is utilized in the transducer 130 depicted in FIG. 1). Preferably, the sensor 110 is a negative temperature coefficient (NTC) thermistor (a ceramic semiconductor). Although the NTC thermistor may be in a form of a glass bead, disc, chip or the like, it is preferred that the NTC thermistor is a bead of about 0.005 inches in diameter. The NTC thermistor's relatively large change in electrical resistance in relation to increasing temperature provides for rapid response to temperature changes, which is particularly advantageous in patient safety feedback methods described below in reference to FIGS. 4-7. Furthermore, the small size of the NTC thermistor mitigates the obstruction to ultrasound energy, yielding improved acoustic imaging quality.

FIG. 2A depicts an exemplary configuration of the acoustic window 120 depicted in FIG. 1, comprising a single sensor in one-layer configuration. In this configuration, the sensor 110 is embedded in the one-layer acoustic window 250. It is to be noted that sensor 110 is positioned in accordance with the objectives described in connection with FIG. 1 above, i.e., to mitigate interference to the transmission of acoustic energy through the one-layer acoustic window 250 and to measure the temperature of the hotspot in the one-layer acoustic window 250. In this configuration, the one-layer acoustic window 250 is a castable plastic, such as RTV (room temperature vulcanizing) silicone, urethane or epoxy. The sensor 110 may be embedded into the castable plastic during the manufacturing process by utilizing a suitable mold, properly positioning the sensor within the mold cavity and injecting or pouring liquid window material into the mold cavity. A hard plastic may also be employed in this configuration. The sensor 110 may be embedded into the hard plastic typically by injection molding during the manufacturing process. A flexible circuit 140 interconnects the sensor 110 via the electrical contact to the controller (or processor) as will be described hereinafter with reference to FIG. 3. It is contemplated that other materials that provide proper acoustic properties for transmission of acoustic energy and are sufficiently durable to the environmental factors, such as polyurethane, may be employed for the one-layer acoustic window 250.

FIG. 2B depicts another exemplary configuration of the acoustic window 120 depicted in FIG. 1, comprising a single sensor in a multi-layer configuration. In this configuration, the acoustic window comprises an outer layer 210, a core 220, the sensor 110 and the flexible circuit 140. The outer layer 210 provides the patient contact surface for contacting the patient during an ultrasound imaging session. The outer layer 210 is made of an environmentally durable material, preferably an impervious polymer material, such as polyethylene, polyester or polyimide. The core layer 220 is an elastomeric material (i.e., “elastomer”) chosen on the basis of its acoustic properties, i.e., acoustic impedance and sound speed. Generally, any thermoplastic elastomer may be employed. It is preferred that the elatomer is a SEBS (styrene-ethylene-butylene-styrene) or a PEBAX (poly-ether-block-amide). The sensor 110 is positioned between the core layer 220 and the outer layer 210. The outer layer 210 provides an environmental barrier that isolates the core layer 220 from the outside environment. It is to be noted that the core layer 220 may also be a castable plastic or a hard plastic, as described with reference to FIG. 2A above. The flexible circuit 140 interconnects the sensor 110 via the electrical contact to the controller (or processor) as will be described hereinafter with reference to FIG. 3.

FIG. 2C depicts still another exemplary configuration of the acoustic window 120 depicted in FIG. 1, comprising a single sensor in another multi-layer configuration. In this configuration, the acoustic window comprises the outer layer 210, the core 220, the sensor 110, the flexible circuit 140, and an inner layer 230. As before, the outer layer 210 provides the patient contact surface for contacting the patient during an ultrasound imaging session. The outer layer 210 and the core layer 220 are made of materials described with reference to FIG. 2B above. The inner layer 230 is made of a polyester material, such as Mylar by Dupont Teijin Films. However, in this configuration, the sensor 110 is positioned between the inner layer 230 and the core layer 220. The inner layer 230 and the outer layer 210 provide an environmental barrier that isolates the core layer 220 from the outside environment, as well as from the internals of the transducer 130 depicted in FIG. 1. The core layer 220 may also be a castable plastic or a hard plastic, as described with reference to FIG. 2A above. The flexible circuit 140 interconnects the sensor 110 via the electrical contact to the controller (or processor) as will be described hereinafter with reference to FIG. 3.

FIG. 2D depicts yet another exemplary configuration of the acoustic window 120 depicted in FIG. 1, comprising a plurality of sensors in single-layer configuration. In this configuration, the sensors 110A, 110B are embedded in the one-layer acoustic window 250. The temperature sensors 110A, 110B are positioned in the one-layer acoustic window 250 in accordance the objectives described in connection with FIG. 1 above, i.e., to mitigate interference to the transmission of acoustic energy through the one-layer acoustic window 250 and to measure the temperature of the hotspot in the one-layer acoustic window 250. Although two sensors are depicted in FIG. 2D, it is contemplated that any number of sensors may be provided based on the particular requirements. The acoustic window is a castable plastic or a hard plastic, as described with reference to FIG. 2A above. Although for clarity and brevity, one flexible circuit 140 is depicted as interconnecting sensor 110A via the electrical contact 240 to the controller (or processor) 310 as depicted in FIG. 3 below, it is to be understood that an additional flexible circuit would be provided for sensor 110B and for any additional sensors provided. As aforementioned, it is contemplated that other materials that provide proper acoustic properties for transmission of acoustic energy and are sufficiently durable to the environmental factors may be employed for the one-layer acoustic window 250, as particularly described with reference to FIG. 2A. The temperature measurements from the plurality of sensors 110A, 110B may be averaged to provide an average temperature.

Alternatively, the highest temperature reading of a sensor among the plurality of sensors 110A, 110B may be used as the temperature measurement. Finally, a primary sensor may be used for temperature measurement with other sensors of a plurality of sensors 110A, 110B providing redundancy for temperature measurement in case sensor malfunction.

FIG. 2E depicts a further exemplary configuration of the acoustic window 120 depicted in FIG. 1, comprising a plurality of sensors in a multi-layer configuration. In this configuration, the acoustic window comprises an outer layer 210, a core 220, sensors 110A and 110B, and the flexible circuit 140. The outer layer 210 provides the patient contact surface for contacting the patient during an ultrasound imaging session. The outer layer 210 and the core layer 220 are made of materials as described with reference to FIG. 2B. The sensors 110A, 110B are positioned between the outer layer 210 and the core layer 220. The outer layer 210 provides an environmental barrier that isolates the core layer 220 from the outside environment. As described above with reference to FIG. 2D, the plurality of sensors 110A, 110B may be averaged to provide an average temperature. Alternatively, the highest temperature reading of a sensor among the plurality of sensors 110A, 110B may be used as the temperature measurement. Finally, a primary sensor may be used for temperature measurement with other sensors of a plurality of sensors 110, 110B providing redundancy for temperature measurement in case sensor malfunction.

FIG. 2F depicts a still further exemplary configuration of the acoustic window 120 depicted in FIG. 1, comprising a plurality of sensors in another multi-layer configuration. In this configuration, the acoustic window comprises the outer layer 210, the core 220, sensors 110A and 110B, the flexible circuit 140, and the inner layer 230. The outer layer 210 provides the patient contact surface for contacting the patient during an ultrasound imaging session. The outer layer 210 and the core layer 220 are made of materials described with reference to FIG. 2B. The inner layer 230 is made of a material described with reference to FIG. 2C. The sensors 110A, 110B are positioned between the inner layer 230 and the core layer 220. The inner layer 230 and the outer layer 210 provide an environmental barrier that isolates the core layer 220 from the outside environment, as well as from the internals of the transducer 130 depicted in FIG. 1. As described with reference to the FIG. 2D above, the temperature measurements from the plurality of sensors 110A, 110B may be averaged to provide an average temperature. Alternatively, the highest temperature reading of a sensor among the plurality of sensors 110A, 110B may be used as the temperature measurement. Finally, a primary sensor may be used for temperature measurement with other sensors of a plurality of sensors 110A, 110B providing redundancy for temperature measurement in case sensor malfunction.

FIG. 3 depicts an exemplary ultrasound medical imaging system comprising the ultrasound medical transducer described with reference to FIGS. 1-2F. A power supply 320, under control of controller 310, supplies power to the various components of the ultrasound medical imaging system. The transducer 130 is also under the control of controller 310. For example, controller 310 controls the amount of power sent to the transducer 130, thereby controlling the power imaging modes of the transducer. The controller includes memory 330 for storing the various programming instructions to control the imaging system. Instructions for the patient safety feedback methods described below in connection with FIGS. 4-7 are stored in memory 330. The controller further executes the instructions in memory 330 to implement the methods described below with reference to FIGS. 4-7. It is noted that the memory 330 may be located anywhere in the imaging system. As aforementioned, the present invention is not limited to any particular type of imaging ultrasound system, nor to any particular imaging or non-imaging mode. The user interface 340 allows the user (i.e., “sonographer”) to monitor the condition of, and/or to interact with, the ultrasound system; it may comprise a keyboard (and mouse), a microphone (and voice recognition software), a handheld input device, or any other form of input device. The presentation means 350 is for presenting system parameters, and may or may not be used to see the results of the ultrasound testing being performed. The presentation means 350 may comprise a display monitor, a speaker (with either voice synthesis or pre-recorded speech), or any other means for presenting the status of system parameters and/or allowing the sonographer to interact with the ultrasound system.

FIG. 4 depicts an exemplary flowchart of a patient safety feedback method that resets an imaging mode at a predetermined temperature within the ultrasound medical imaging system depicted in FIG. 3. In step 410, the controller 310 monitors the measurements from sensor 110 of the current temperature T_{cur} at the patient contact surface of acoustic window 120 of the ultrasound transducer 130. In step 410, it is assumed that the transducer 130 is in the highest power imaging mode (i.e., “original imaging mode”). It is to be noted that if more than one sensor is used, such as sensors 110A, 110B of FIGS. 2D-2F, the highest temperature measurement may be used as current temperature T_{cur} or the measurements may be averaged to obtain current temperature T_{cur}. In step 420, it is determined whether the current temperature T_{cur} has exceeded a threshold temperature Tₜₕ. The threshold temperature Tₜₕ may be any temperature less than or equal to a critical temperature T_{crit} at which discomfort and/or damage may occur to the patient being imaged. If the current temperature T_{cur} is below the threshold temperature Tₜₕ in step 420, the method returns to the monitoring step 410. If the current temperature T_{cur} is greater than or equal to the threshold temperature Tₜₕ in step 420, the method switches to a lower power imaging mode at step 430. Because of the lower power generated in the lower power imaging mode, the transducer 130 does not become as hot, and will be able to cool from the higher temperature caused by the previous highest power imaging mode. The switching to the lower power imaging mode in step 430 may be accomplished by changing (rather than reducing) one or more system parameters in order to reduce the temperature in step 430. The one or more system parameters to be changed may be predetermined, i.e., hardwired or programmed into the imaging system. Alternatively, the one or more parameters to be changed may be input by the sonographer, who may select and determine the amount of change of one or more of the system parameters.

Further with reference to FIG. 4, it is determined if the imaging system should be reset to the original imaging mode in step 440. The reset is determined based on whether the current temperature T_{cur} is less than or equal to the reset temperature Tᵣₑₛ, which is below the threshold temperature Tₜₕ. If it is determined that the current temperature T_{cur} is less than or equal to the reset temperature Tᵣₑₛ in step 440, the imaging system is switched back to the original imaging mode in step 450. The reset to the original imaging mode may be accomplished by switching the one or more system parameters that were changed previously in step 430 back to their original values, as set before their change in step 430. Alternatively, if it is determined that the reset in step 440 is not necessary, the method continues monitoring the current temperature T_{cur} until the condition in step 440 is met and the reset to the original imaging mode is performed in strep 450. Thereafter, the method of FIG. 4 continues with the monitoring in step 410. Other reset methods are possible, such as, a manual reset performed by the sonographer. The system parameters that can be changed in order to decrease the transducer temperature are described in U.S. Pat. No. 6,709,392, which is incorporated herein by reference in its entirety. The system may use any of these reset methods or a combination of these reset methods, as desired or as a particular circumstance may demand. Lastly, it is to be noted that in some circumstances the imaging system may not reset to the original imaging mode and remains in the lower power imaging mode until the sonographer completes the imaging session.

FIG. 5 depicts an exemplary flowchart of a patient safety feedback method that resets the imaging mode via user input within the ultrasound medical imaging system depicted in FIG. 3. The steps of FIG. 5 that are the same as those in FIG. 4 perform the same functionality; they are incorporated herein in their entirety and therefore will not be described. In FIG. 5, step 440 has been replaced by step 540 in which the method monitors the imaging system for user input to determine whether the sonographer has indicated a switch from the lower power imaging mode back to the original power imaging mode. If it is determined that the sonographer has indicated a switch in step 540, the method switches the ultrasound imaging system to the original imaging mode in step 450. The sonographer therefore has the ability to switch to the highest power imaging mode (original imaging mode) in step 450. The method of FIG. 5 continues with monitoring in step 410. Although, not depicted for clarity and brevity, it is understood that the sonographer likewise has the ability to switch out of the original imaging mode in step 450 to the lower power imaging mode in step 430. In this manner, the sonographer may avoid reaching threshold temperature Tₜₕ by changing to a lower power imaging mode whenever the original power imaging mode is not necessary or when the sonographer ascertains that the temperature is getting too hot.

FIG. 6 depicts a flowchart of a patient safety feedback method that resets the imaging mode to a fundamental imaging mode or reduces a transmit voltage of the ultrasound medical transducer if the imaging mode is already set to the fundamental imaging mode within the ultrasound medical imaging system depicted in FIG. 3. More specifically, the power imaging mode defaults to the fundamental imaging mode once the threshold temperature Tₜₕ is reached. If the system is already in the fundamental imaging mode, the system reduces the transmit voltage in order to reduce the temperature of the ultrasound transducer 130. It is to be noted that in step 410 the imaging system is in the original power imaging mode. The temperature T_{cur} is monitored in step 410, and in step 420 it is determined if the current temperature T_{cur} is greater than or equal to the threshold temperature Tₜₕ. If the current temperature T_{cur}, is less than the threshold temperature Tₜₕ in step 420, the method continues monitoring by returning to step 410. If the current temperature T_{cur} is greater than or equal to the threshold temperature Tₜₕ in step 420, the method continues with step 625.

Further with reference to FIG. 6, it is determined whether the imaging system is currently in the fundamental imaging mode in step 625. If the imaging system is not in the fundamental imaging mode in step 625, the system switches to the fundamental imaging mode in step 630. Thereafter, it is determined whether the imaging system should be reset to the original power imaging mode in step 635. In this context, the reset step 635 covers any manner of resetting the imaging system to the original power imaging mode. Thus, step 635 may be the automatic determining step 440 described in reference to FIG. 4, the user-controlled determining step 540 described in reference to FIG. 5, or any other type of resetting. If it is determined that the imaging systems is to be reset in step 635, the method continues with step 640 in which the imagining system is switched back to the original imaging mode, and the method returns to the monitoring step 410.

Still further with reference to FIG. 6, if the system is in fundamental imaging mode in step 625, the method reduces the transmit voltage of the imaging system in step 645. In step 650, it is determined whether the imaging system should be reset to the original power imaging mode, in similar fashion as described in reference to reset step 635. If it is determined that the imaging systems is to be reset in step 650, the method continues with step 655 in which the imagining system is switched back to the original imaging mode, and the method returns to monitoring in step 410. It is to be noted that the reduction in transmit voltage of the imaging system in step 645 may be replaced by a step that changes other parameters of the imaging system in order to reduce the ultrasound transducer temperature. These system parameters include, but are not limited to: the duty cycle (the system will reduce the amount of time the transmitting piezoelectric elements are active during each transmit-receive cycle), frequency (the system decreases the frequency of the ultrasound sound waves), frame rate (the system decreases the frame rate, i.e., number of sweeps per second), pulse repetition frequency (“PRF”—the system decreases the number of beams formed per second), aperture (the system decreases the size of the aperture), imaging depth (the system decreases the scanning depth), and/or sector width (the system decreases the width of the zone being scanned).

FIG. 7 depicts an exemplary flowchart of a patient safety feedback method that sets or resets the imaging mode via user input. The controller 310 monitors the measurements from sensor 110 of the current temperature T_{cur} at the patient contact surface of acoustic window 120 of the ultrasound transducer 130. In step 410, it is assumed that the transducer 130 is in the highest power imaging mode (i.e., “original imaging mode”). It is to be noted that if more than one sensor is used, such as sensors 110A, 110B of FIGS. 2D-2F, the highest temperature measurement may be used as current temperature T_{cur} or the measurements may be averaged to obtain current temperature T_{cur}. This monitored temperature is displayed on the presentation means 350. The sonographer may monitor or observe the temperature and act to change the power imaging mode. In addition to the current temperature T_{cur} of the patient contact surface, other imaging system parameters described above may be also displayed on the presentation means 350. The temperature and other system parameters of information may be displayed in a variety of ways: as gauge icons, as digital readouts, as histograms, or any other manner of visually indicating a quantity. The other system parameters may include the voltage, the current power imaging mode, the frame rate, the sector width, and others. In addition, the time remaining before the transducer reaches the critical temperature T_{crit} may also be displayed so that the sonographer may correctly determine the correct time to change the power imaging modes.

Further with reference to FIG. 7, the method continues with step 420, which determines whether the current temperature T_{cur} is greater than or equal to the threshold temperature Tₜₕ. If the current temperature T_{cur} is greater than or equal to the threshold temperature Tₜₕ, an alarm will sound or issue to indicate that the sonographer should switch power imaging mode before the transducer reaches the critical temperature T_{crit}. The alarm may take any convenient form to attract the sonographer's attention, such as an audio, visual or audiovisual alert. For example, the alarm may generate an audio sound and/or appear as a flashing icon on the presentation means 350. After the alarm has issued in step 725, it is determined whether the sonographer initiated a switch of modes in step 735. If the sonographer did not initiate a switch of modes in step 735, the method returns to step 725 in which the alarm is reissued. If the sonographer initiated a switch of imaging modes in step 735, the imaging mode is switched in step 750 and the method of FIG. 7 returns to monitoring in step 410.

Still further with reference to FIG. 7, if the current temperature T_{cur} is lower than the threshold temperature Tₜₕ in step 420, it is determined whether the sonographer has initiated a switch of imaging modes in step 740. If the sonographer did not initiate a switch of modes in step 740, the method returns to step 410 in which the method monitors the current temperature T_{cur}. If the sonographer initiated a switch of imaging modes in step 740, the imaging mode is switched in step 750 and the method of FIG. 7 returns to monitoring in step 410.

It is to be noted that the manner in which the images formed from the different imaging modes are combined for display to the user can take a variety of forms. Examples of display formats include: alternating frames, alternating scan lines, or compound image.

In the alternating frames format, the display alternates frames between images based on harmonic imaging and images based on fundamental imaging. This mixed format produces a flickering appearance, which is not too distracting because the overall effect of the image is as if it was being illuminated by different lighting (not simply alternating bright and dim illumination). This mixed format can be assisted by automatically adjusting the brightness of the harmonic image to that of the fundamental image. In the alternating scan lines format, every other line in the scanned plane is scanned with the harmonic image. The resulting combined image is displayed with the regular display averaging which is used to smooth and fill in scan lines. This averaging of the regular display smoothes the appearance of the image. In the compound image format, a compound image is formed by displaying a central harmonic image of limited width and filling out the edges of the sector with the low power, fundamental mode image.

The present invention is in no way limited to the embodiments described above and more complex methods are contemplated. As one example, although 3D imaging is not explicitly discussed, the imaging system could be easily applied to 3D imaging. As another example, any of the methods above could be modified by the addition of extra steps in which the ultrasound transducer is turned off once the critical temperature T_{crit} (at which patient discomfort or damage may occur) is reached. As yet another example, various levels of threshold alerts may also be generated as the ultrasound transducer temperature comes closer to the critical temperature T_{crit}.

It will be understood that the temperature at the patient contact surface as measured by the temperature sensor(s) located in the acoustic window enhance the ultrasound imaging system and the patient safety methods described hereinabove. Since the temperature measurement is a precise determination of the acoustic window temperature, rather than an estimate of such temperature, patient safety and comfort are under more strict control, and may be more rapidly adjusted in accordance with the patient safety methods described above.

While there have been shown and described fundamental novel features of the invention as applied to preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A medical ultrasound transducer comprising:
an acoustic window (120) for contacting a patient at a patient contact surface for imaging the patient; and
at least one temperature sensor (110, 110A, 110B) located in the acoustic window for determining patient contact temperature at the patient contact surface.

2. The medical ultrasound transducer in accordance with claim 1, wherein the at least one sensor is selected from the group consisting of: a negative temperature coefficient thermistor; a thermocouple; a resistance temperature detector; and a fiber optic sensor using thermalchromic liquid crystals.

3. The medical ultrasound transducer in accordance with claim 1, wherein the acoustic window comprises at least one layer (210, 220, 230, 250).

4. The medical ultrasound transducer in accordance with claim 3, wherein the at least one layer is a layer selected from the group consisting of: a thermoplastic elastomer; a castable plastic; and a hard plastic.

5. The medical ultrasound transducer in accordance with claim 4, wherein the thermoplastic elastomer is a styrene-ethylene-butylene-styrene (SEBS) or poly-ether-block-amide (PEBAX).

6. The medical ultrasound transducer in accordance with claim 4, wherein the castable plastic is selected from the group consisting of: room temperature vulcanizing (RTV) silicone; urethane; and epoxy.

7. The medical ultrasound transducer in accordance with claim 3, wherein the at least one layer is an impervious polymer selected from the group consisting of: polyethylene; polyester; and polyimide.

8. The medical ultrasound transducer in accordance with claim 3, wherein the sensor is embedded into the at least one layer (250).

9. The medical ultrasound transducer in accordance with claim 3, wherein the at least one layer includes an inner layer (230) and a core layer (220), and the sensor (110) is placed between the inner layer and the core layer.

10. The medical ultrasound transducer in accordance with claim 1, wherein the at least one sensor is located in one position selected from the group consisting of: about the center of the acoustic window; offset from about the center of the acoustic window; about the edge of the acoustic window; and about the hotspot of the acoustic window.

11. A medical ultrasound imaging system comprising:
an ultrasound transducer (130) including (i) an acoustic window (120) for contacting a patient at a patient contact surface for imaging the patient; and (ii) at least one temperature sensor (110, 110A, 110B) located in the acoustic window for determining patient contact temperature at the patient contact surface; and
a controller (310) for controlling a power imaging mode of the ultrasound transducer in accordance with the determined patient contact temperature.

12. The medical ultrasound imaging system in accordance with claim 11, further comprising:
a presentation means (350) for displaying the imaging of the patient; and
a user interface (340) for receiving input from a sonographer to the controller.

13. The medical ultrasound imaging system in accordance with claim 11, further comprising:
a patient safety feedback means (110, 310, 320, 330) for (i) monitoring patient contact temperature from the at least one sensor (410), and (ii) switching between a lower power imaging mode and a higher power imaging mode based on the monitored patient contact temperature (430, 450).

14. The medical ultrasound imaging system in accordance with claim 12, further comprising a patient safety feedback means (110, 310, 320, 330, 340, 350) for (i) monitoring patient contact temperature from the at least one sensor (410), (ii) displaying the monitored patient contact temperature to the sonographer, (iii) receiving input via the user interface from the sonographer (540), and (iv) switching between a lower power imaging mode and a higher power imaging mode based on the received input from the sonographer (450).

15. The medical ultrasound imaging system in accordance with claim 11, wherein the at least one sensor (110, 110A, 110B) of the ultrasound transducer (130) is selected from the group consisting of: a negative temperature coefficient thermistor; a thermocouple; a resistance temperature detector; and a fiber optic sensor using thermalchromic liquid crystals.

16. The medical ultrasound imaging system in accordance with claim 11, wherein the acoustic window of the ultrasound transducer comprises at least one layer (210, 220, 230, 250).

17. The medical ultrasound imaging system in accordance with claim 16, wherein the at least one layer is a layer selected from the group consisting of: a thermoplastic elastomer; a castable plastic; and a hard plastic.

18. The medical ultrasound imaging system in accordance with claim 17, wherein the thermoplastic elastomer is a styrene-ethylene-butylene-styrene (SEBS) or poly-ether-block-amide (PEBAX).

19. The medical ultrasound imaging system in accordance with claim 17, wherein the castable plastic is selected from the group consisting of: room temperature vulcanizing (RTV) silicone; urethane; and epoxy.

20. The medical ultrasound imaging system in accordance with claim 16, wherein the at least one layer is an impervious polymer selected from the group consisting of: polyethylene; polyester; and polyimide.

21. The medical ultrasound imaging system in accordance with claim 16, wherein the at lest one the sensor is embedded into the at least one layer (250).

22. The medical ultrasound imaging system in accordance with claim 16, wherein the at least one layer includes an inner layer (230) and a core layer (220), and the at least one sensor (110, 110A, 110B) is placed between the inner layer and the core layer.

23. The medical ultrasound imaging system in accordance with claim 11, wherein the at least one sensor of the ultrasound transducer is located in one position selected from the group consisting of: about the center of the acoustic window; offset from about the center of the acoustic window; about the edge of the acoustic window; and about the hotspot of the acoustic window.

24. A method for imaging a patient using a medical ultrasound imaging system, the imaging system comprising an ultrasound transducer including an acoustic window having a patient contact surface and at least one temperature sensor located in the acoustic window, the method comprising:
(a) contacting the patient contact surface of the acoustic window to the patient for imaging the patient;
(b) determining patient contact temperature of the ultrasound transducer at the patient contact surface from the at least one temperature sensor; and
(c) controlling a power imaging mode of the ultrasound transducer in accordance with the determined patient contact temperature.

25. The method for imaging a patient in accordance with claim 24, further comprising:
displaying the determined patient contact temperature to a sonographer;
receiving input from the sonographer based on the displayed patient contact temperature; and
controlling the power imaging mode of the ultrasound transducer in accordance with the received input from the sonographer.
